(19) United States
(12) Patent Application Publication
(10) Pub. No.: US 2007/0295329 A1
(43) Pub. Date: Dec. 27, 2007

(54) Dose indicator

(76) Inventors: Eric A. Lieberman, Scotch Plains, NJ (US); Erick Rios, Atlanta, GA (US); Devin Moore, Decatur, GA (US)

Correspondence Address: MICHAEL W. HAAS;RESPIRONICS, INC., 1010 MURRY RIDGE LANE, MURRYSVILLE, PA, 15668, US

(21) Appl. No.: 11/811,648

(22) Filed: Jun. 11, 2007

Related U.S. Application Data
(60) Provisional application No. 60/813,212, filed on Jun. 13, 2006.

(51) Int. Cl. B67D 5/08; A61M 11/02; B67D 5/06

(52) U.S. Cl. 128/200.23; 206/459.1; 222/36

(57) ABSTRACT

An apparatus and method for tracking the amount of medicament remaining with a metered dose inhaler. A dose indicator for a metered dose inhaler includes a housing having an upper portion and a lower portion, a spring element structured to moveably connect the upper portion with the lower portion, and a control module received within the housing and adapted to index a count representative of the number of doses of medicament remaining within the metered dose inhaler each time the dose indicator is cycled.

### CROSS-REFERENCE TO RELATED APPLICATIONS

This application claims priority under 35 U.S.C. §119(e) from provisional U.S. patent application No. 60/813,212 filed Jun. 13, 2006 the contents of which are incorporated herein by reference.

### BACKGROUND OF THE INVENTION

1. Field of the Invention

The present invention generally relates to tracking the amount of a medicament within a metered dose inhaler and, more particularly, to a dose indicator for indexing and displaying a count representative of the number of doses of a medicament within a metered dose inhaler.

2. Description of the Related Art

A metered dose inhaler (MDI) is employed to deliver a dose of aerosolized medicament directly to a patient's lungs. An MDI may be employed, for example, to treat asthma, chronic obstructive pulmonary disease (COPD), and other respiratory problems. Typically, an MDI includes a boot and a pressurized canister. The boot has a mouthpiece end and a canister end. The pressurized canister, containing the medicament, is inserted into the canister end of the boot. The patient, when administering a dose of medicament, places the mouthpiece end of the boot into their mouth, actuates the MDI, and breathes in the aerosolized medicament. The MDI is actuated (i.e., “fired”) by pressing down on the canister.

Generally, the canister includes a metering valve, a metering chamber, and a reservoir. The metering valve includes a stem and a spring. At rest, the spring biases the stem such that the metering valve is in a closed position. When in the closed position, the metering chamber is connected by a flow path in the stem to the reservoir which contains the medicament. Thus, medicament from the reservoir fills the metering chamber. In the closed position, however, the stem isolates the metering chamber from the ambient atmosphere, thus preventing medicament from being discharged from the metering chamber to the ambient atmosphere. As the stem is depressed into the container against the spring, the stem passes through an intermediate position at which the metering chamber is isolated from both the reservoir and the ambient atmosphere. Further travel of the stem, places the metering valve in the open position. In the open position, the metering chamber is connected by a flow path in the stem to the ambient atmosphere. The metering chamber, however, is isolated from the reservoir. Accordingly, medicament is discharged from the metering chamber to the ambient atmosphere, but additional medicament from the reservoir is not discharged. When the downward pressure is removed from the canister, the spring causes the stem to travel from the open position, through the intermediate position, and back to the closed position (thus allowing the medicament from the reservoir to re-fill the dosage chamber).

Because an MDI is commonly used to treat asthma, chronic obstructive pulmonary disease (COPD), and other respiratory problems, there are numerous situations where it is necessary or desirable to know how many doses of medicament remain within the canister. To withstand pressurization, however, the canister is usually constructed from metal. As a result, the amount of medicament within the canister cannot be determined visually. Current methods for determining the amount of medicament remaining within the canister are inaccurate, inconvenient, wasteful, and inadequate.

Waste, for instance, occurs when the patient over-counts the number of doses that were dispensed and discards the canister with medicament remaining therein. A more serious problem may occur, however, when the patient undercounts the number of doses that were dispensed. As a result, the patient may believe that additional doses of medicament remain within the canister when, in fact, the canister is empty. Undercounting may lead to serious consequences. For example, asthma sufferers who have undercounted the number of doses previously dispensed from their MDI may be exposed to an increased risk of a severe asthma attack because their MDI does not have sufficient medicament remaining to treat an attack in its initial stages.

Different types of dose indicators have been created to maintain a count representative of the number of doses remaining within the MDI. These dose indicators, however, are inaccurate, unreliable, and too large. Many of these dose indicators also require modifications to the MDI. Accordingly, there is a need for a dose indicator that overcomes these and other problems associated with tracking the amount of a medicament remaining within the MDI.

### SUMMARY OF THE INVENTION

Accordingly, one aspect of the present invention is directed to a dose indicator which comprises a housing, a spring element, and a control module. The housing has an upper portion and a lower portion which are moveably connected by the spring element. The control module is received within the housing and is adapted to index a count each time the dose indicator is cycled.

Another aspect of the present invention is directed to a drug delivery system which comprises a metered dose inhaler and a dose indicator. The metered dose inhaler includes a boot and a canister. The canister is inserted into the boot and is adapted to hold a medicament therein. The canister includes a stem which is structured to cause the canister to release a dose of the medicament when the metered dose inhaler is fired. The dose indicator is attachable to the canister and is structured to indicate a count representative of an amount of medicament within the canister. The dose indicator comprises a housing having an upper portion and a lower portion, a spring element structured to moveably connect the upper portion with the lower portion, and a control module received within the housing and adapted to index the count each time the dose indicator is cycled.

Another aspect of the invention is directed to a method for tracking an amount of medicament within in a canister for a metered dose inhaler. The method comprises programming a count representative of a number of doses of medicament within the canister into a dose indicator, connecting the dose indicator to the canister, detecting when the dose indicator is cycled, and, responsive to the detecting, indexing the count. The dose indicator comprises a housing having an upper portion and a lower portion, a spring element structured to moveably connect the upper portion with the lower portion, and a control module received within the housing and adapted to index the count each time the dose indicator is cycled.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 illustrates a dose indicator according to one embodiment of the present invention coupled with a metered dose inhaler.

FIG. 2 is a perspective view of the dose indicator of FIG. 1.

FIG. 3 is a side view of the dose indicator of FIG. 2.

FIG. 4 is an exploded view of the dose indicator of FIG. 2.

FIG. 5 is a perspective view of the lower portion of the housing for the dose indicator of FIG. 2.

FIG. 6 is a perspective view of the control module for the dose indicator of FIG. 2.

FIG. 7 is a perspective view of the spring element for the dose indicator of FIG. 2.

FIG. 8 is a perspective view of the base portion for the dose indicator of FIG.2.

FIG. 9 is an exploded view of a first sub-assembly for the dose indicator of FIG. 2

FIG. 10 is a perspective view of the first sub-assembly of FIG. 9.

FIG. 11 is an exploded view of a bottom assembly for the dose indicator of FIG. 2.

FIG. 12 is a perspective view of the bottom assembly of FIG. 11.

FIG. 13 is a perspective view of a second sub-assembly for the dose indicator of FIG. 2.

FIG. 14 is an exploded view of a top assembly for the dose indicator of FIG. 2.

FIG. 15 is a perspective view of the top assembly of FIG. 14.

FIG. 16 is a bottom, perspective view of a breakaway member for the dose indicator of FIG. 1.

FIG. 17 is a top view of the breakaway member of FIG. 16.

FIG. 18 is a perspective view of an attachment member for the dose indicator of FIG. 1.

FIG. 19 is a flow chart illustrating an operational process for employing the dose indicator of FIG. 1.

FIG. 20 is a flow chart illustrating an operational process for employing the dose indicator of FIG. 1 according to an alternative embodiment.

### DETAILED DESCRIPTION OF THE EXEMPLARY EMBODIMENTS

Directional phrases used herein, such as, for example, left, right, clockwise, counterclockwise, top, bottom, up, down, and derivatives thereof, relate to the orientation of the elements shown in the drawings and are not limiting upon the claims unless expressly recited therein.

As employed herein, the term “number” shall mean one or more than one and the singular form of“a”, “an”, and “the” include plural referents unless the context clearly indicates otherwise.

As employed herein, the statement that two or more parts are “connected” or “coupled” together shall mean that the parts are joined together either directly or joined together through one or more intermediate parts. Further, as employed herein, the statement that two or more parts are “attached” shall mean that the parts are joined together directly.

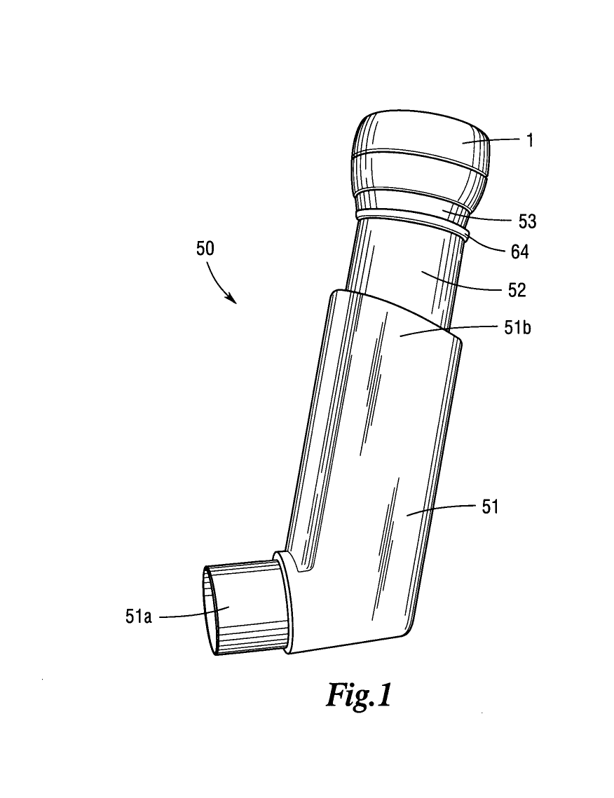

FIG. 1 illustrates a metered dose inhaler 50 (MDI). MDI 50 is coupled with a dose indicator 1 which is constructed according to the principles of the present invention. MDI 50 includes a boot 51 and a canister 52. The boot has a mouthpiece end 51a and a canister end 51b. In the current embodiment, a breakaway member 53 and an attachment member 64 are employed to releasably couple dose indicator 1 with canister 52. It is contemplated, however, that another manner of coupling dose indicator 1 with MDI 50 may be used while remaining within the scope of the present invention. As seen in FIG. 1, dose indicator 1 does not restrict the air flow around canister 52 and/or the air flow into canister end 51b of boot 51. As a result, the air flow to the patient (e.g., exiting mouthpiece end 51a) and/or the dispensed medicament's particle size are not negatively affected by the use of dose indicator 1.

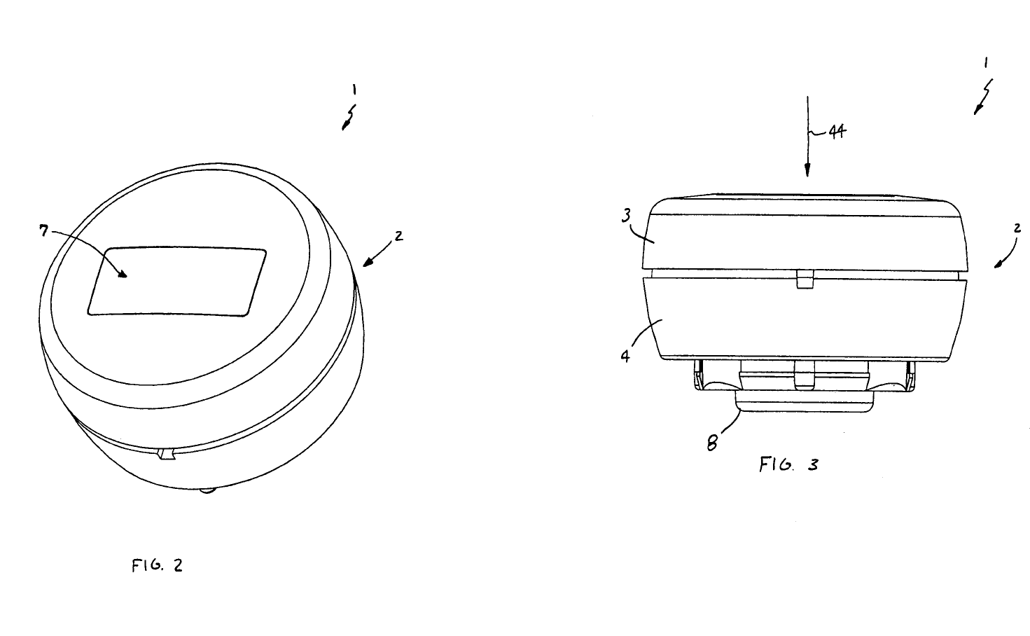

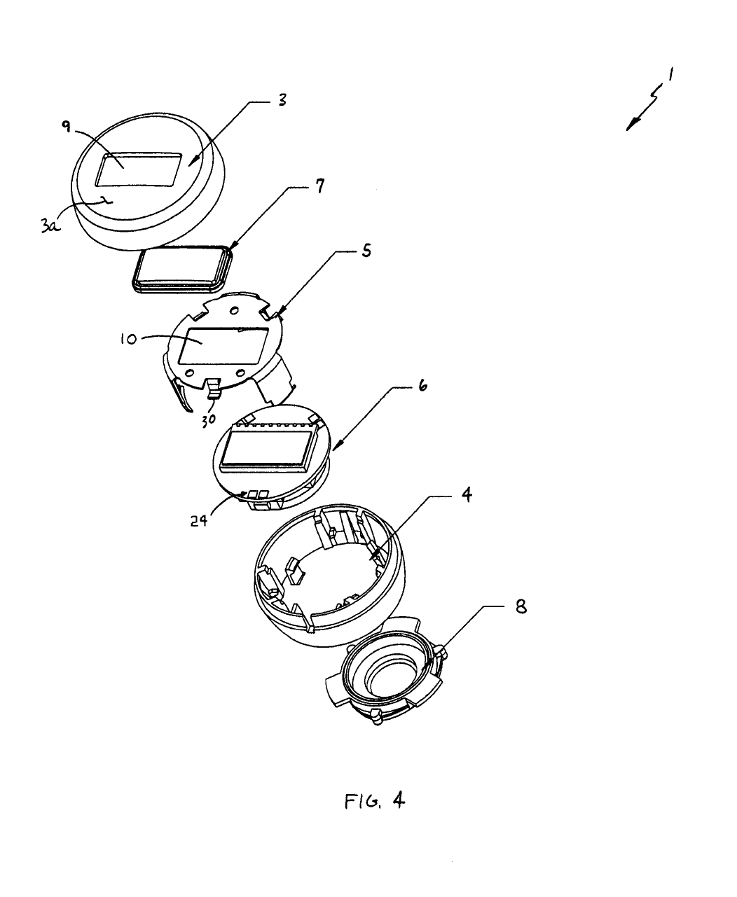

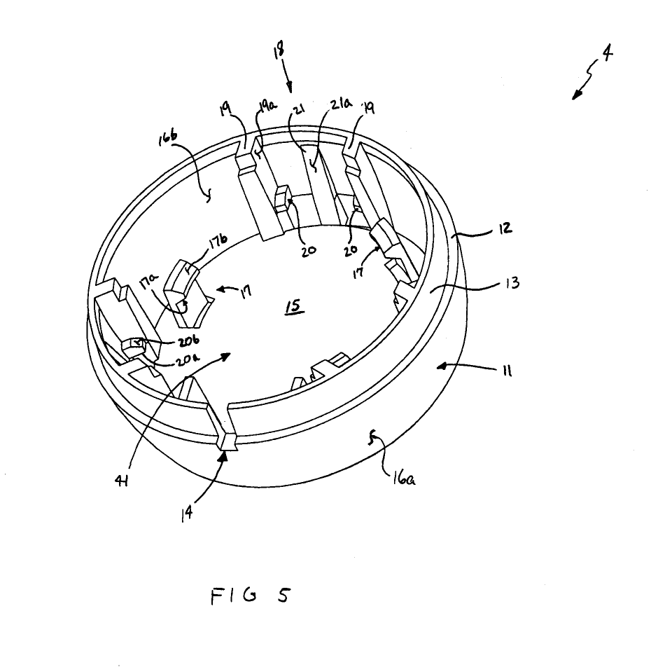

FIGS. 2-4 are perspective, side, and exploded views illustrating an exemplary embodiment of dose indicator 1 according to the principles of the present invention. As discussed above, dose indicator 1 is adapted for use with MDI 50. More specifically, dose indicator 1 is adapted to couple with MDI 50 and, each time MDI 50 is fired, index a count representative of the number of doses of a medicament within MDI 50.

Dose indicator 1 includes a housing 2, having an upper portion 3 and a lower portion 4, a spring element 5, and a control module 6. In the current embodiment, dose indicator 1 also includes a lens element 7 and a base portion 8.

Generally, control module 6 is contained within housing 2. Upper portion 3 and lower portion 4 of housing 2 are moveably connected by spring member 5. In the current embodiment, spring member 5 is fixed to top portion 3, but moveably retained in lower portion 4. Housing 2 and spring member 5 cooperate such that spring member 5 generates an upward force which biases upper portion 3 away from lower portion 4.

Spring member 5 includes a number of contacts 30. Each contact 30 is associated with a contact pad pair 24 on control module 6. In the current embodiment, upper portion 3 is biased away from lower portion 4 such that contacts 30 normally do not bridge (i.e., electrically connect) their associated contact pad pair 24 (i.e., dose indicator 1 is said to be in an “open” state). When a sufficient downward force (as indicated by directional arrow 44) is exerted on upper portion 3, however, one of more of contacts 30 may bridge their associated contact pad pairs 24 (i.e., dose indicator 1 is said to be in a “closed” state).

As stated above, spring element 5 is structured to moveably connect upper portion 3 with lower portion 4 such that dose indicator 1 may be cycled between states. Control module 6 is adapted to index a count each time dose indicator 1 is cycled. As employed herein, the term “cycle”, and derivatives thereof, refers to a designated number of state changes for representing that a dose of medicament has been released from MDI 50. For example, in the current embodiment, dose indicator 1 is structured such that substantially the same amount of force is needed to fire MDI 50 as is need to cause dose indicator 1 to change from the open state to the closed state. Thus, in the current embodiment, a “cycle” generally refers to changing dose indicator 1 from the open state to the closed state (i.e., dose indicator 1, which is normally in the open state, is structured to represent that a dose of medicament has been released from MDI 50 when dose indicator 1 changes from the open state to the closed state). Depending on the specific application, however, a “cycle” may refer to changing, for example and without limitation, from the open state to the closed state, from the closed state to the open state, from the open state to the closed state and back to the open state, and/or from the closed state to the open state and back to the closed state.

A detailed view of lower portion 4 according to the principles of the present invention is illustrated in FIG. 5. Lower portion 4 includes a generally circular bottom 15 with a wall 11 extending upward from the outer circumference thereof. Bottom 15 and wall 11 define a cavity 41. Wall 11 includes an outer surface 1 6a and an inner surface 16b. Outer surface 16a includes a step 12 and a lip 13 at the top thereof. An orifice 14 is structured to provide access to cavity 41 through wall 11.

Lower portion 4 also includes a number of spring member retaining slots 18 on inner wall 16b. In the current embodiment, three retaining slots 18 are evenly spaced (i.e., at substantially 120° apart) about inner wall 16b. Each retaining slot 18 includes a ramp 21 disposed between two pillars 19. Ramp 21, as illustrated in FIG. 5, is at its minimum thickness near the top of wall 11 and at its maximum thickness near bottom 15 (i.e., ramp 21 slopes towards the center of cavity 41 as it nears bottom 15). Each pillar 19 includes a spring retaining member 20 located on an inner surface 19a thereof.

Lower portion 4 also includes a number of catches 17 extending upwardly into cavity 41 from bottom 15. Each catch 17 includes a retaining surface 17a and a sloped surface 17b. In the current embodiment, three catches 17 are evenly spaced (i.e., at substantially 120° apart) about an inner circumference of bottom 15. As will be discussed in greater detail below, catches 17 are structured to couple lower portion 4 to control module 6. It is contemplated that a different number of catches and/or alternative spacing arrangement may be employed while remaining within the scope of the present invention. Furthermore, it is contemplated that another suitable structure and/or manner of coupling control module 6 and lower portion 4 may be employed.

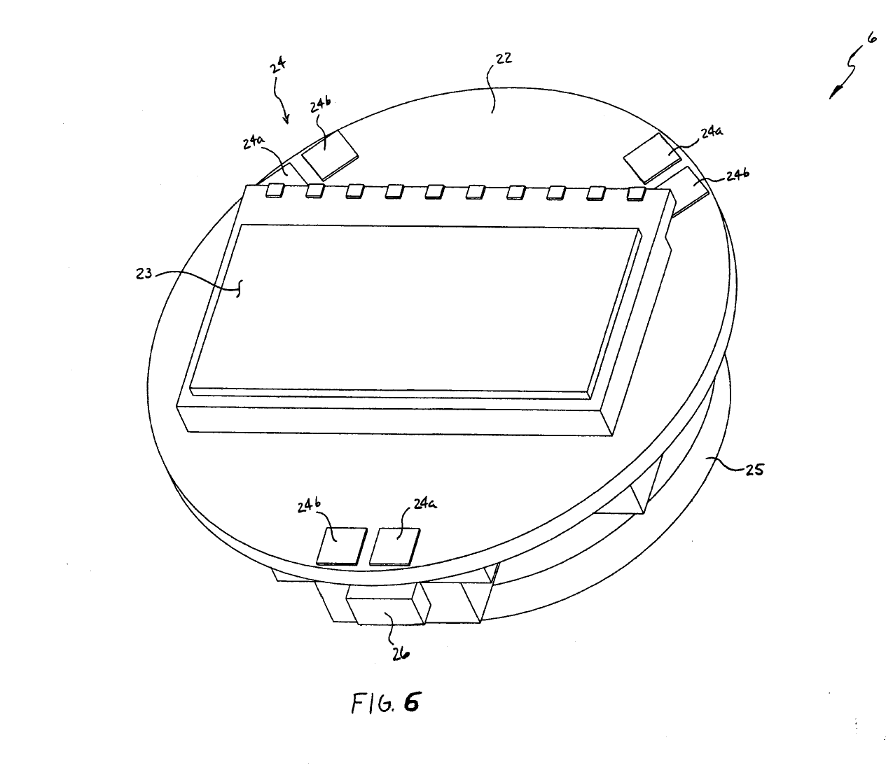

Referring now to FIG. 6, control module 6 includes a pedestal 25, a printed circuit board (PCB) 22, and a display 23. In the current embodiment, pedestal 25 carries PCB 22, which in turn, carries display 23. The PCB 22 includes a number of contact pad pairs 24 and a central processing unit (CPU) (not shown). Contact pads pairs 24 are space around an outer periphery of the top surface of PCB 22. Each contact pad pair 24 includes a first contact pad 24a and a second contact pad 24b. Each contact pad 24a, 24b is operatively coupled to the CPU. The CPU is adapted, for example and without limitation, to sense whether the contact pad pairs 24 are bridged or un-bridged (i.e., whether a contact pad 24a and a contact pad 24b are electrically connected or electrically disconnected). In response to the sensing, the CPU is adapted to execute a number of routines for, without limitation, tracking the amount of medicament within a metered dose inhaler. In the current embodiment, the CPU is a general purpose LCD controller, for example, part number HT49C30-1 manufactured by Holtek Semiconductor, Inc.

A button 26 is operatively connected to the PCB 22 and may be employed to provide input to the CPU. Button 26, for example and without limitation, may be used to initially set the count, manually index the count, set alarm levels, and acknowledge an alarm. A battery (not shown), for supplying power to the control module 6, is placed between pedestal 25 and PCB 22.

It is contemplated that the number of contact pad pairs 24, their specific arrangement on the PCB 22, and the type and number of inputs, among others, may be altered while remaining within the scope of the present invention. For example, contact pad pair 24 may be comprised of two electrical traces encircling the outer perimeter of the PCB 22 which are bridgeable by one or more contacts 30. As another example, contact pad pairs 24 may be replaced with stand alone contact pads, each of which are electrically connected to a first terminal of the battery. Associated contacts 30, electrically connected to a second terminal of the battery, are structured to engage its associated stand alone contact pad and completed an electrical circuit which is detectable by the CPU.

Additionally, it is contemplated that any CPU structured to execute a number of routines for, without limitation, keeping a count representative of the amount of medicament within a metered dose inhaler may be employed while remaining with the scope of the present invention.

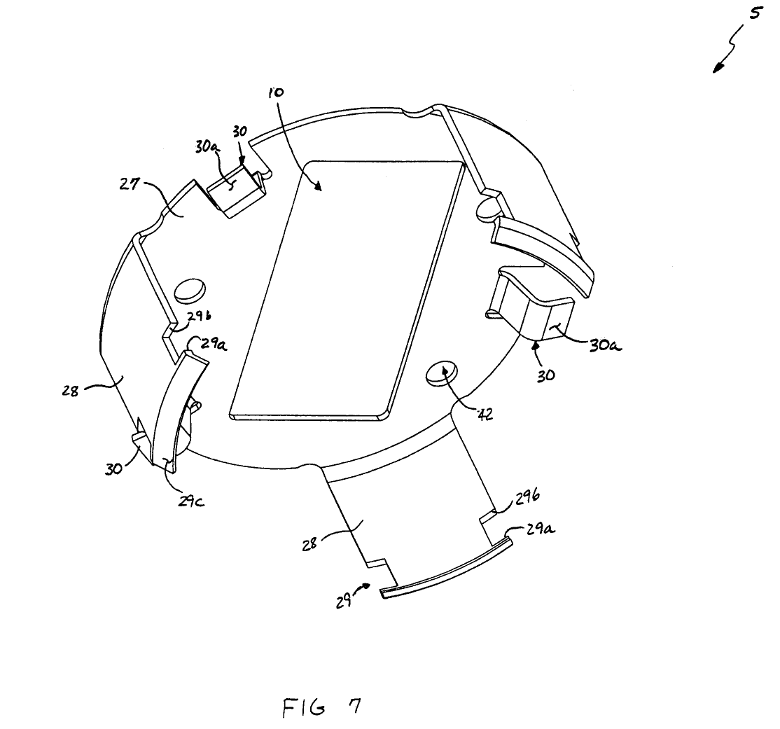

Referring to FIG. 7, spring element 5 includes a base plate 27 having a number of spring arms 28 and a number of contacts 30 extending therefrom. Each spring arm 28 has an angled leading edge 29c, which generally slopes towards the center of base plate 27. Each spring arm 28 also includes a number of notches 29 therein. Each notch has a retaining surface 29a and a stop surface 29b. Contacts 30 are structured to bridge a corresponding contact pad pair 24 on PCB 22. Each contact 30, as will be discussed in more detail below, includes a surface 30a which is adapted to electrically connect the pads of a corresponding contact pad pair 24.

Base plate 27 also includes an aperture 10 therein. Aperture 10 is generally rectilinear in shape and is structured to allow display 23 to be viewed through lens element 7 when dose counter 1 is assembled. In the current embodiment, spring element 5 is formed from a single piece of stamped metal with spring arms 28 and contacts 30 being formed therefrom. When inserted into slots 18, spring arms 28 flex inwards towards the center of base plate 27 thereby placing spring arms 28 under tension.

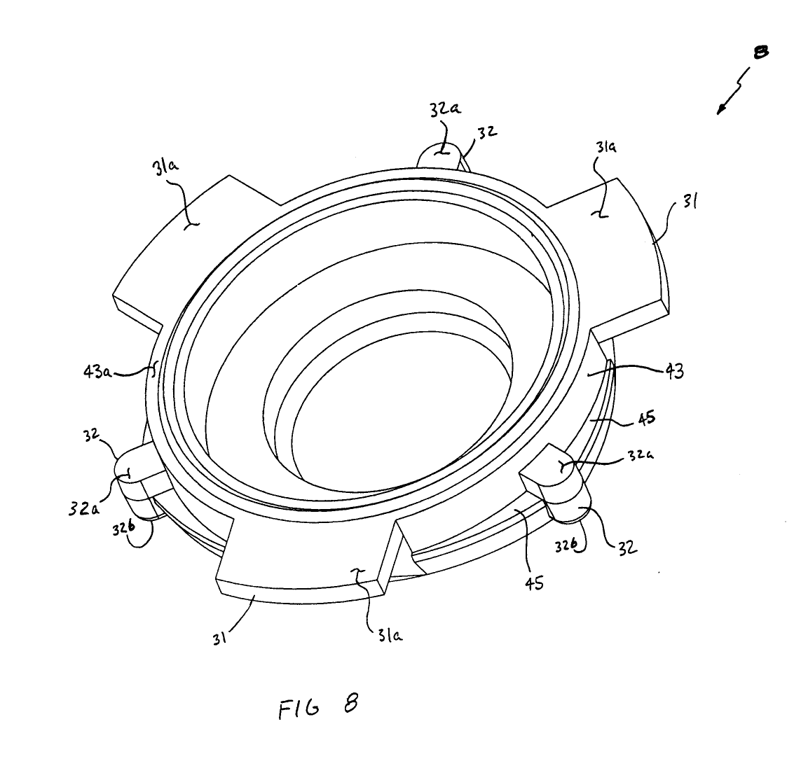

FIG. 8 illustrates base portion 8 for dose counter 1. In the current embodiment, base portion 8 includes three tabs 31 and three posts 32 spaced evenly in an alternating fashion around wall 43 of base portion 8. As seen in FIG. 8, a top surface 3 la of each tab 31 is substantially even within top surface 43a of wall 43; whereas posts 32 are slightly offset below top surface 43a. Each post 32 includes webs 45 extending from the sides thereof. Each web 45 tapers from post 32 towards a tab 31.

Referring briefly to FIG. 9, the bottom of lower portion 4 and bottom of base portion 81 are shown. Lower portion 4 includes a ring 34 having a number of recesses 33, each structured to receive a corresponding tab 31 from base portion 8 therein. Additionally, surfaces 34a of ring 34 are structured to abut with surfaces 32a of posts 32.

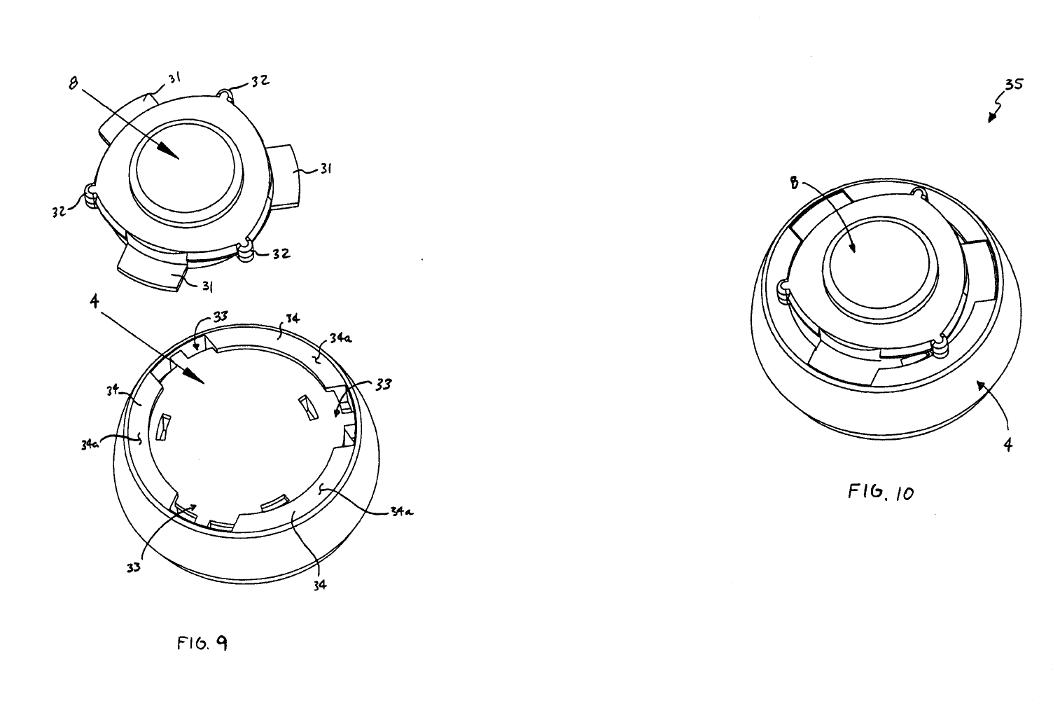

Assembly of dose indicator 1 according to one embodiment will now be discussed. A first sub-assembly 35 (FIG. 10) is formed by coupling base portion 8 with lower portion 4. More specifically, base portion 8 is oriented with the bottom of lower portion 4 such that tabs 31 are aligned with corresponding recesses 33 and posts 32 are aligned with corresponding surfaces 34a. Once aligned, base portion 8 is ultrasonically welded to lower portion 4 thereby creating first sub-assembly 35. Base portion 8 is structured to help seal any openings in lower portion 4 created, for example, during the manufacturing process. Although first sub-assembly 35 is illustrated as being formed from two components, it should be noted that any number of components may be used to create a first sub-assembly. For example, a first-subassembly of unitary construction is contemplated.

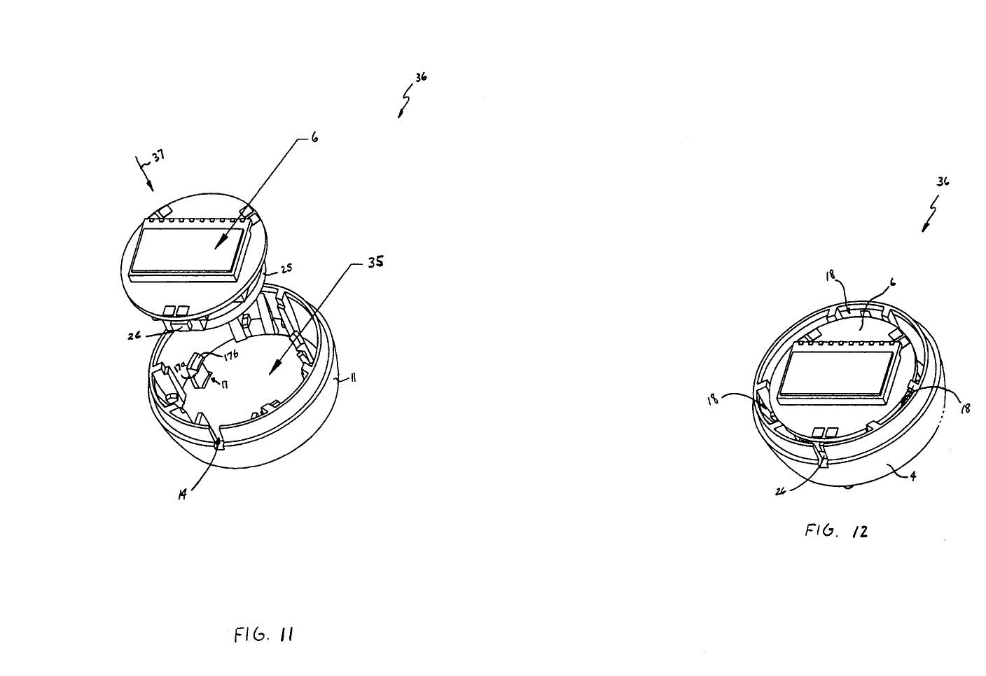

After first sub-assembly 35 is completed, control module 6 is coupled with first sub-assembly 35 to obtain a bottom assembly 36. Referring to FIG. 11, control module 6 is oriented with first sub-assembly 35 such that button 26 is in alignment with orifice 14. Control module 6 is then inserted into cavity 41 such that the bottom of pedestal 25 contacts slope surfaces 17b of catches 17. Control module 6 is then pressed down further into cavity 41 as indicated by directional arrow 37. This downward force causes catches 17 to flex slightly towards outer wall 11 and allows control module 6 to “snap” into place. Once control module 6 snaps into place, retaining surfaces 17a engage the top of pedestal 25 thereby coupling control module 6 with first sub-assembly 35. As best seen in FIG. 12, button 26, although contained within cavity 41, is accessible from outside bottom assembly 36 via orifice 14. It should further be noted from FIG. 12 that slots 18 also remain accessible after control module 6 is coupled to first sub-assembly 35.

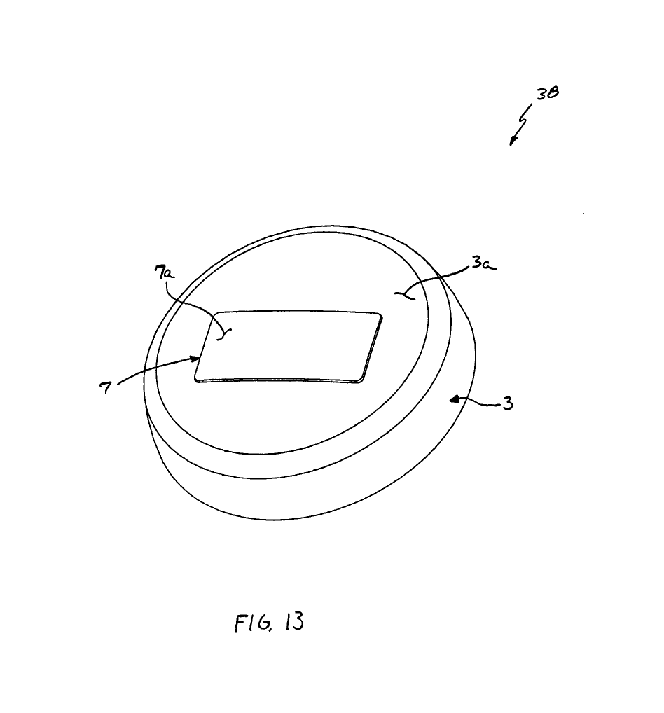

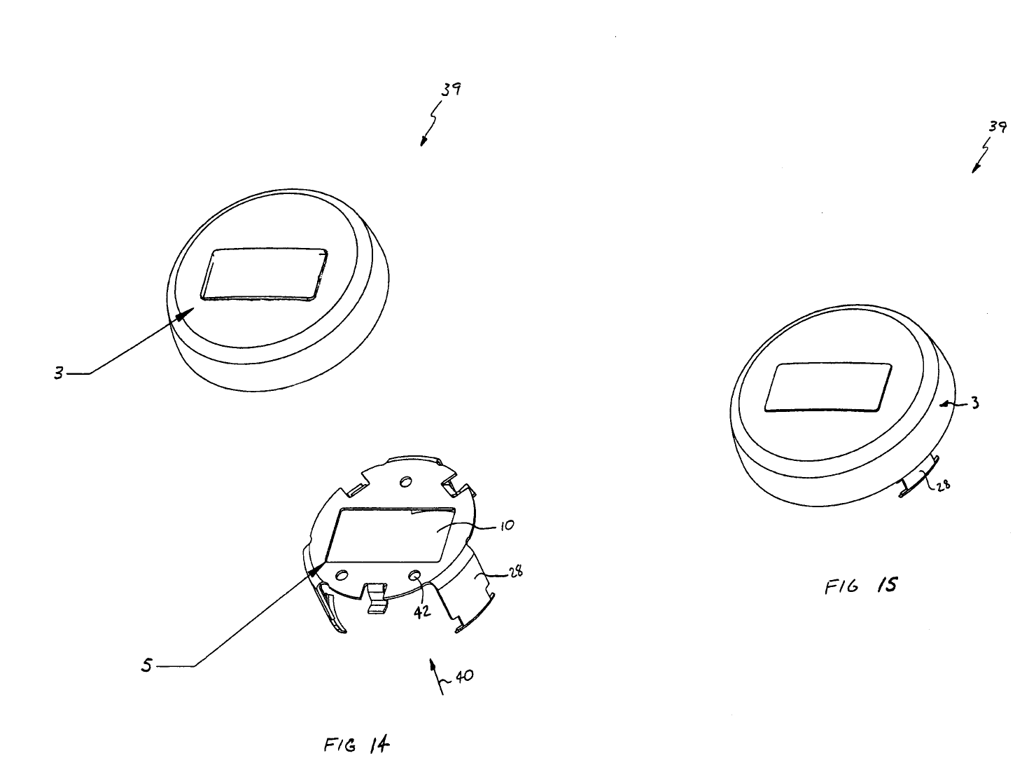

A second sub-assembly 38 (FIG. 13) is formed by coupling lens element 7 with upper portion 3. In the current embodiment, a portion of lens element 7 is inserted through aperture 9 from the bottom side of upper portion 3. Lens element 7 is then ultrasonically welded to upper portion 3. As shown in FIG. 13, lens element 7 is structured such that, when coupled with upper portion 3, a top surface 7a of lens element 7 is substantially flush with a top surface 3a of upper portion 3.

After second sub-assembly 38 is completed, spring element 5 is coupled with second sub-assembly 38 to create a top assembly 39. Referring to FIG. 14, spring element 5 is oriented such that aperture 10 aligns with lens element 7. Spring element 5 is then moved upward, as indicated by directional arrow 40, until a bottom surface (not shown) of lens element 7 is inserted into aperture 10. Spring element 5 is then thermally staked to second sub-assembly 38, for example through punch-outs 42, to complete top assembly 39. It should be noted that spring arms 28 extend down from upper assembly 39 as illustrated in FIG. 15.

Top assembly 39 and bottom assembly 36 are then connected to form dose indicator 1 (FIGS. 2-3). In the current embodiment, top assembly 39 is oriented such that spring arms 28 are in alignment with corresponding slots 18, which as discussed above, are accessible in bottom assembly 36. Top assembly 39 and bottom assembly 36 are then brought together such that spring arms 28 enter into slots 18. Each spring arm 28, once within a corresponding slot 18, contacts sloped surface 21a of ramp 21. Sloped surface 21a begins to deflect spring arm 28 away from outer wall 11. This deflection places spring arm 28 under tension. Accordingly, spring arms 28 begin to generate an upward force on top assembly 39 relative to bottom assembly 36.

As spring arm 28 travels deeper into its corresponding slot 18, leading edge 29c contacts sloped surfaces 20b of spring retaining members 20. Sloped surfaces 20b cause spring arm 28 to deflect even farther away from outer wall 11. Once leading edge 29c passes sloped surfaces 20b, spring arm 28 “snaps” into place. More specifically, spring retaining member 20 enters into notches 29 and retaining surfaces 20a of spring retaining members 20 engage retaining surface 29a of notches 29. In this position, top assembly 39 is coupled with bottom assembly 36. In the current embodiment, this position (i.e., when retaining surface 20a is engaged with retaining surface 29a) may be referred to as the “open state” because contact surfaces 30a have not yet bridged the pads 24a, 24b of any of the contact pad pairs 24.

The continued application of downward pressure, as indicated by directional arrow 44 in FIG. 3, will cause spring arms 28 to travel deeper into their corresponding slots 18 and will cause a contact surface 30a of at least one of contacts 30 to bridge pads 24a, 24b of its associated contact pad pair 24. In the current embodiment, this position may be referred to as the “closed state” because a contact surface 30a has bridged pads 24a, 24b of at least one contact pad pair 24. Continuing the application of downward pressure (after contacts 30 have bridged contact pad pairs 24), may cause spring arms 28 to travel a slight distance further within slots 18 and may cause contacts 30 to flex. At a certain point, however, upper portion 3 comes into contact with step 12 of lower portion 4 and/or stop surface 29b comes into contact with spring retaining member 20, thus halting any further travel of spring arms 28 within slots 18.

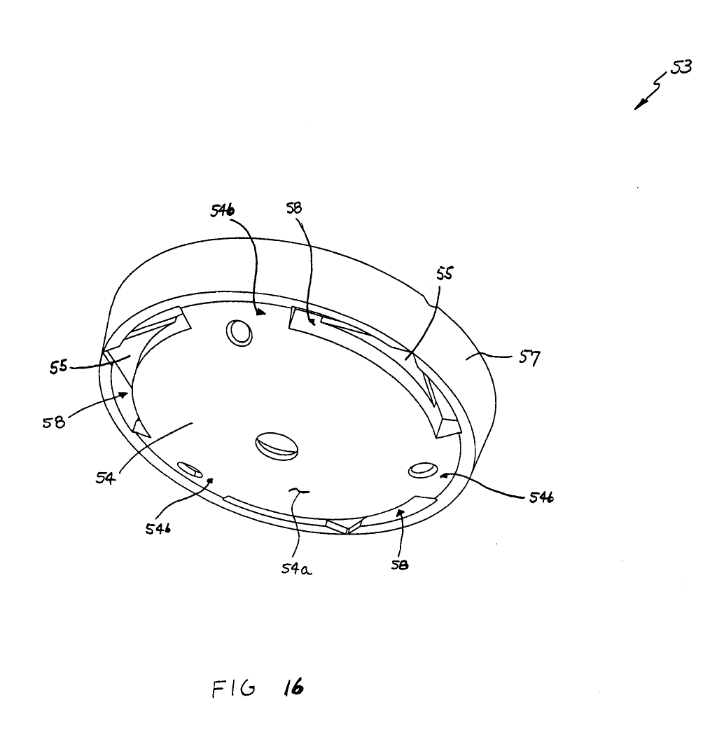

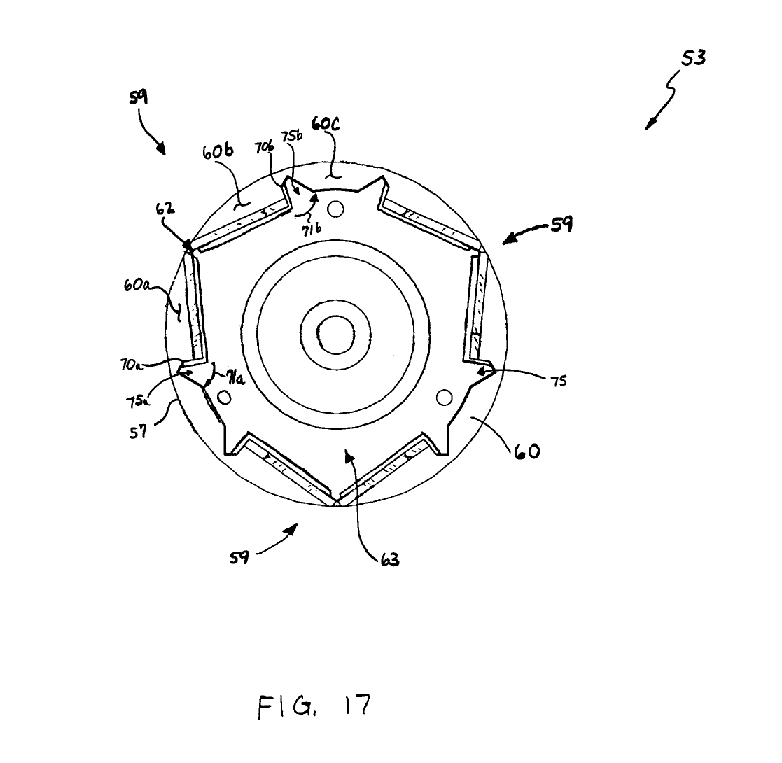

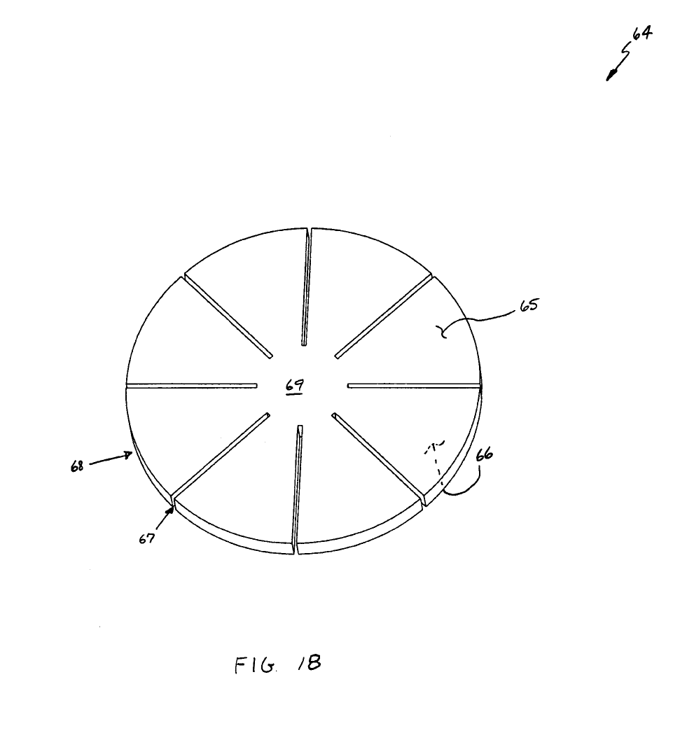

As discussed above in conjunction with FIG. 1, dose indicator 1 is coupled with MDI 50 by a breakaway member 53 (FIGS. 16-17) and an attachment member 64 (FIG. 18). Attachment member 64 includes a first surface 65 and a second surface 66, each having an adhesive thereon. First surface 65 is attached to a bottom surface 54a of breakaway member 53, typically during the manufacturing process. Second surface 66 is covered with a peel-away covering (not shown) which can be removed by a patient prior to attaching attachment member 64 and breakaway member 53 to canister 52.

Attachment member 64 is adapted to conform to bottom surface 54a of breakaway member 53 and to canister 52. For example, attachment member 64 includes a number of slits 67 extending radially outward from a center portion 69. The slits 67 define a number of pie-shaped segments 68 which can move independently relative to each other such that attachment member 64 better conforms to the shape of bottom surface 54a and to canister 52.

It is contemplated that breakaway member 53 and attachment member 64 will be discarded when canister 52 is empty. Accordingly, breakaway member 53 is structured to breakably couple with dose indicator 1 in the current embodiment. Referring to FIG. 16, breakaway member 53 includes a substantially round base 54 with a side wall 57 extending upward from the outer circumference thereof. In the current embodiment, side wall 57 extends from a number of spokes 54b in base 54. Spokes 54b and sidewall 57 define a number of openings 58. A number of triangular vanes 55 radially extend from an inner circumference of base 54 to partially occlude an associated opening 58 and to further connect base 54 with side wall 57. In the current embodiment, the thickness of side wall 57 is reduced at or near the points where side wall 57 and vanes 55 are connected (i.e., at each corner 62). As will be discussed in more detail below, an upper surface (not shown) of vanes 55 are structured to carry surface 32b of post 32 on base portion 8.

Referring to FIG. 17, the top of side wall 57 includes a lip 60. Side wall 57 and lip 60 define a cavity 63. In the current embodiment, lip 60 can generally be sub-divided into three segments 59. Each segment 59 has a first portion 60a and a second portion 60b, which meet each other at a corner 62. Each segment 59 is separated from each other segment 59, by a third portion 60c and notches 75.

Cavity 63 is structured to receive base portion 8 of dose indicator 1 therein. Generally, dose indicator 1 is oriented such that each post 32 of base portion 8 is aligned substantially with an associated corner 62. Base portion 8 is then pressed into cavity 63. Lip 60 is slightly deflected by webs 45 until base portion 8 “snaps” into breakaway member 53 (i.e., base portion 8 is coupled with breakaway member 53). More specifically, webs 45 are engaged by first portion 60a and second portion 60b such that base portion 8 is retained within cavity 63 of breakaway member 53. When engaged, surfaces 32b (of posts 32) are typically carried by the upper surfaces of vanes 55.

When base portion 8 is coupled with breakaway member 53, first portion 60a and second portion 60b, which abut an associated post 32, prevent rotation of base portion 8 relative to breakaway member 53. Base portion 8, however, can be uncoupled from breakaway member 53 by applying to base portion 8 a rotational force that is sufficient to cause first portion 60a and/or second portion 60b to fracture. For example, when base portion 8 is rotated counter-clockwise with sufficient force, post 32 causes first portion 60a to break at or near corner 62. Once broken, first portion 60a rotates about pivot point 70a (as indicated by directional arrow 71a) and into notch 75a. Webs 45 then disengage from first portion 60a and second portion 60b; thus, base portion 8 can be withdrawn from cavity 63. Likewise, when base portion 8 is rotated clockwise with sufficient force, post 32 causes second portion 60b to break at or near corner 62. Once broken, second portion 60b rotates about pivot point 70b (as indicated by directional arrow 71b) and into notch 75b. Webs 45 then disengage from first portion 60a and second portion 60b; thus, base portion 8 can be withdrawn from cavity 63. Accordingly, dose indicator 1 can be un-coupled from breakaway member 53 and re-used with another breakaway member 53 which has been coupled with a full canister 52. Although discussed in the context of first portion 60a and/or second portion 60b breaking, it is contemplated that a portion of side wall 57 may also break when base portion 8 is being separated from breakaway member 53. For example, it is contemplated that side wall 57 may break at its point of reduced thickness (i.e., at corner 62).

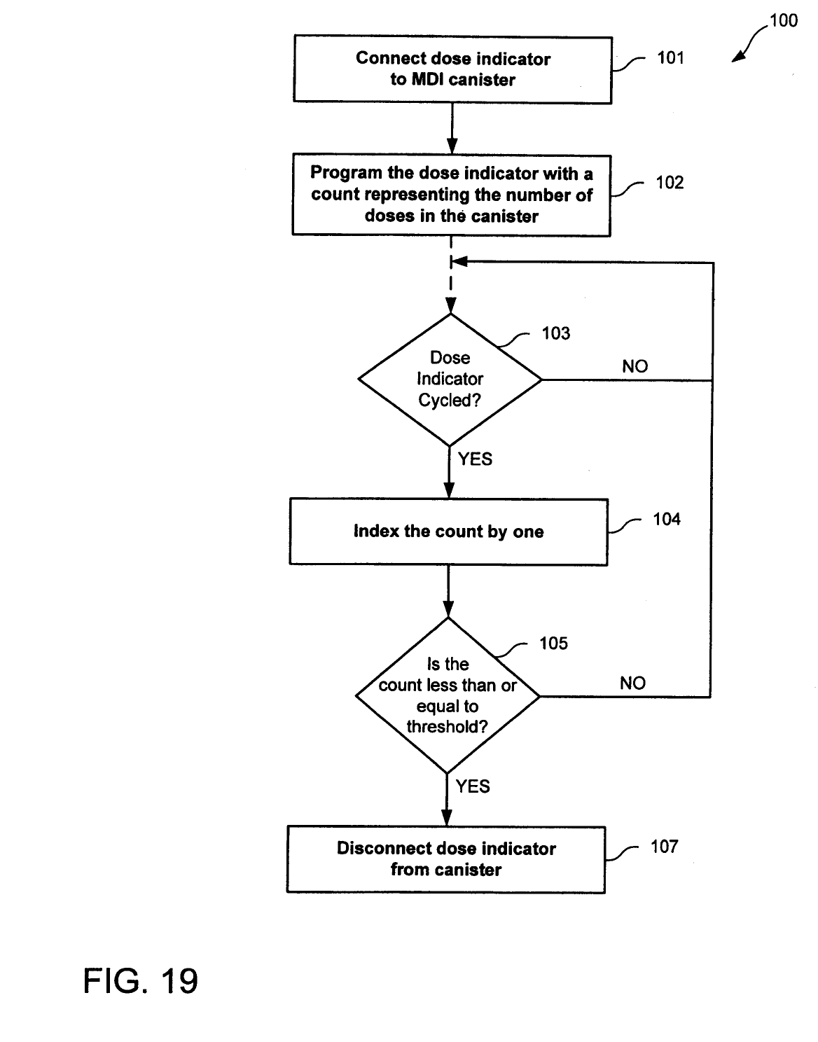

The general operation of dose indicator 1 according to the principles of the present invention will now be discussed in conjunction with operational process 100 (FIG. 19). Operational process 100 is initiated when dose indicator 1 is connected to canister 52 of MDI 50. In the current embodiment, dose indicator 1 is connected to a full canister 52 using breakaway member 53 and attachment member 64. For example, a patient removes a peel-away covering on the bottom of attachment member 64 and attaches attachment member 64 and breakaway member 53 to canister 52. Dose indicator 1 is then coupled with breakaway member 53 as discussed above in conjunction with FIGS. 16 and 17.

Operational control then passes to operation 102 wherein a count representative of the number of doses of medicament within the full canister 52 is programmed into dose indicator 1. Generally, the number of doses contained within the full canister 52 is marked on the outside of the canister 52. In the current embodiment, this count is programmed into the control module 6, for example using button 26. This count is indicated on display 23 for viewing by the patient. In the current embodiment, the count is continuously displayed, however, it is contemplated that control module 6 may have a sleep mode such that the count is not displayed after a certain amount of inactivity. It is further contemplated that the order of operations 102 and 101 may be reversed (i.e., the count programmed prior to attaching dose indicator 1 to canister 52).

After the count is programmed into dose indicator 1, operation process 100 is suspended until pressure is applied to the dose indicator 1, for example, a pressure applied to fire MDI 50. At operation 103, a determination is made as to whether the dose indicator 1 has been cycled. If dose indicator 1 has not been cycled, control branches “NO” such that operational process 100 loops back to operation 103. If dose indicator 1 has been cycled, control branches “YES” and operation 104 assumes control.

In the current embodiment, dose indicator 1 is structured to cycle whenever dose indicator 1 is switched from the open state to the closed state. More specifically, dose indicator 1 is structured such that slots 18 cause spring arms 28 to flex thereby generating an upward force on top assembly 39 relative to bottom assembly 36. As a result, contacts 30 are held such that they do not bridge contact pads 24a, 24b of contact pad pairs 24 when dose indicator 1 is at rest (i.e., dose indicator 1 is normally in the open state). Dose indicator 1 is cycled when at least one contact 30 bridges a corresponding contact pad pair 24 (i.e., whenever dose indicator 1 changes from the open state to the closed state). For example, dose indicator 1 is structured to cycle when a patient applies a sufficient amount of pressure to the top of dose indicator 1 to cause at least one contact 30 to bridge its corresponding contact pad pair 24 (i.e., to enter the closed state).

Dose indicator 1 is structured such that the amount of force required to cause at least one contact 30 to bridge its corresponding contact pad pair 24 is the same as or slightly less than the amount of force required to fire MDI 50. In other words, dose indicator 1 is structure to cycle each time MDI 50 is fired, thus reducing under-counting and/or over-counting. A patient, when administering a dose of medicament for example, places mouthpiece end 51 a into their mouth and presses down on the top of dose indicator 1. This action causes top assembly 39 to travel towards bottom assembly 36 until at least one contact 30 bridges a contact pad pair 24 (i.e., dose indicator 1 is in the closed state). Dose indicator 1 is structured such that the at least one contact 30 bridges its associated contact pad pair 24 at substantially the same moment that MDI 50 is structured to fire. When MDI 50 fires, the patient breathes in the dispensed medicament.

In the current embodiment, contacts 30 and corresponding contact pad pairs 24 are arranged such that at least one contact pad pair 24 is bridged when a sufficient amount of pressure is applied to the top of dose indicator 1, even if the pressure is applied off-center (e.g., even if the applied pressure causes top assembly 39 to “rock” with respect to bottom assembly 36). Although discussed in context of cycling from the open state to the closed state, it is contemplated that dose indicator 1 may be adapted to cycle, for example and without limitation, whenever dose indicator 1 is switched from the closed state to the open state while remaining within the scope of the present invention. For instance, contact pad pairs 24 may be located on the bottom of PCB 22 and contacts 30 may be structured to bridge contact pad pairs 24 without pressure being applied to the top of dose indicator 1 (i.e., normally in the closed state). In this configuration, dose indicator 1 is cycled by applying pressure in the direction indicated by arrow 44 to open the normally closed switch between contacts 30 and contact pad pairs 24.

After dose indicator 1 is cycled in operation 103, operation 104 indexes the count. In the current embodiment, the phrase “indexing the count,” and all derivatives thereof, refer to decrementing the count. For example, control module 6 is adapted to index the count by one each time dose indicator 1 is cycled. Assume for instance that a full canister 52 included twenty-five (25) doses of medicament and that this count (i.e., 25) was programmed into control module 6 in operation 102. Further assume that a determination was made that dose indicator 1 was cycled at operation 103. As a result, the count is indexed by one (i.e., 25−1) and the new count (i.e., 24) is indicated on display 23 at operation 104.

Although discussed in context of decrementing the count by one, it is contemplated that dose indicator 1 may be adapted such that control module 6 increments the count (e.g., from 0 to the total number of doses) while remaining within the scope of the present invention. Accordingly, it is contemplated that “indexing the count” may refer to decrementing and/or incrementing the count by one or any multiple thereof. Furthermore, although Arabic numerals are used to indicate the count in the current embodiment, it is contemplated that, for example and without limitation, any alpha-numeric characters and/or symbols may be employed to indicate the count while remaining within the scope of the present invention.

After the count is indexed at operation 104, a determination is made at operation 105 as to whether the count representative of the number of doses remaining within the canister 52 is less that or equal to a threshold. In the current embodiment, the threshold is set to zero. Accordingly, operational control branches “NO” if the count is greater than zero and operational control is passed back to operation 103; whereas operational control branches “YES” if the count is zero and operational process 100 is terminated at operation 107.

At operation 107, in the current embodiment, dose indicator 1 disconnected from breakaway member 53 (and thus canister 52) as described above in conjunction with FIGS. 16 and 17. In the current embodiment, breakaway member 53 remains connected to canister 52. The patient may then replace the depleted canister 52 with a full canister 52. Operation 100 may then be repeated with the full canister 52 in place.

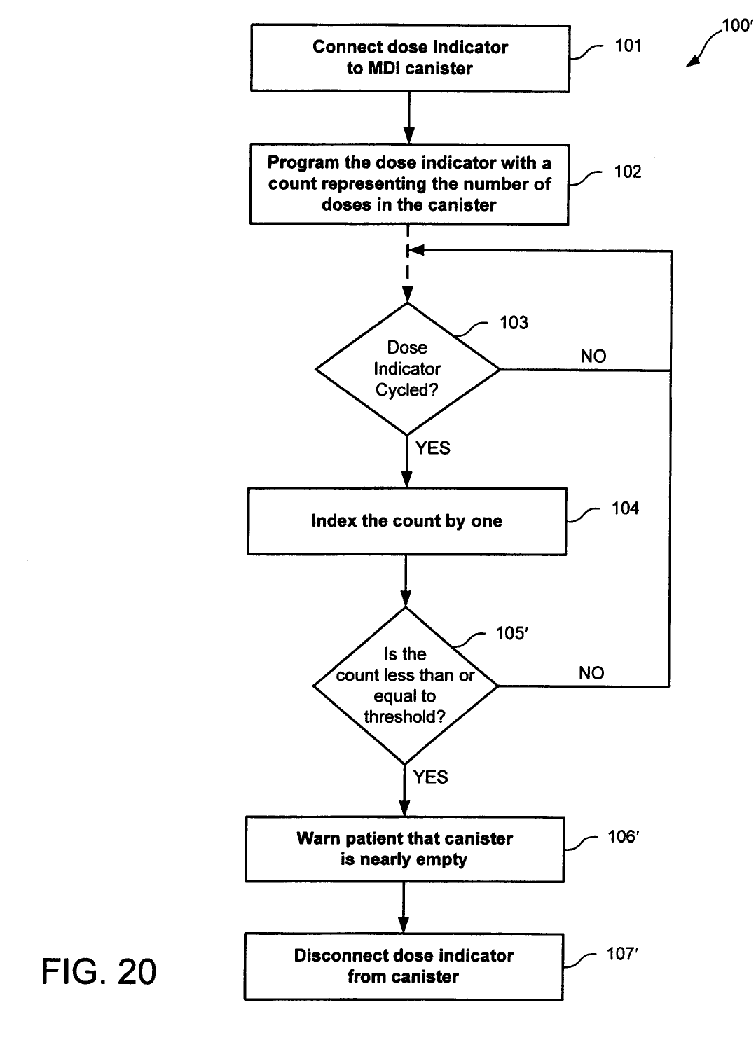

It should be noted that operational process 100 may be modified as needed, for example, to include additional functionality. FIG. 20, for example, illustrates operational process 100′ which provides the operation of dose indicator 1 according to an alternative embodiment of the present invention. Specifically, operational process 100′ provides an alarm to the patient when it is determined that the count representative of the number of doses remaining within the canister is less that or equal to the threshold.

Referring to FIG. 19 and 20, it is noted that operations 101-104 of operational process 100′ are generally the same as operations 101-104 of operational process 100. Accordingly, the detailed discussion of operations 101-104 in conjunction with operational process 100′ will be omitted.

Referring now to FIG. 20, after the count is indexed at operation 104, a determination is made at operation 105′ as to whether the number of doses remaining within the canister 52 is equal to less than or equal to a predetermined threshold. In the current embodiment, the threshold is set such that the patient is given sufficient advance warning that the medicament within canister 52 is nearly depleted so that the patient is afforded an opportunity to obtain a new canister 52 prior to the current canister 52 actually being depleted. For example, the predetermined threshold is-set at five (5) to provide a safety factor for the patient. Accordingly if the count has just been indexed to six (6) in operation 104, control branches “NO” at operation 105′ and control passes back to operation 103. If, however, the count has just been indexed to five (5) in operation 104, control branches “YES” at operation 105′ and control is passed to operation 106′.

At operation 106′, the patient is warned that canister 52 is nearly depleted or is, in fact, empty. Several types of warnings may be used, for example and without limitation, the count indicated on display 23 may flash, the color of display 23 may change, and/or an audible alarm may be emitted.

In operation 107′, the patient disconnects the dose indicator 1 from the canister. In the current embodiment, dose indicator 1 is disconnected from breakaway member 53 (and thus canister 52) as described above in conjunction with FIGS. 16 and 17. Dose indicator 1 is then separated from the breakaway member 53, which remains connected to canister 52. The patient may then replace the depleted canister 52 with a full canister 52. Operation 100′ may then be repeated with the full canister 52 in place.

It is also contemplated that operational process 100′ may be altered while remaining within the scope of the present invention. For example, operational process 100′ may easily be altered such that the patient may deplete the remaining medicament from the current canister 52 prior to disposal (eliminating waste). In the discussion above, the threshold was set such that the patient is given an advanced warning with five (5) doses of medicament remaining within the canister 52. Operational process 100′ may be altered such that the patient can administer these remaining doses. More specifically, operation 100′ may be altered such that the count continues to be indexed each time the dose indicator 1 is subsequently cycled. Operation process 100′ may further be adapted such that each subsequent time the count is indexed, a warning of increasing intensity is provided. For example, display 23 may flash at a count of five (5), may change to red at a count of four (4), may flash and turn red at a count of three (3), may emit an audible warning at a count of two (2), may emit an audible warning and flash at a count of one (1), and may emit an audible warning, flash, and turn red at a count of zero (0).

Although the invention has been described in detail for the purpose of illustration based on what is currently considered to be the most practical and preferred embodiments,:it is to be understood that such detail is solely for that purpose and that the invention is not limited to the disclosed embodiments, but, on the contrary, is intended to cover modifications and equivalent arrangements that are within the spirit and scope of the appended claims. For example, it is contemplated that spring member 5 may be structured as part of bottom assembly 36 and that retaining slots 18 may be structured as a part of top assembly 39. Additionally, it is to be understood that the present invention contemplates that, to the extent possible, one or more features of any embodiment can be combined with one or more features of any other embodiment.

What is claimed is:

1. A dose indicator, comprising:
a housing having an upper portion and a lower portion;
a spring element structured to moveably connect the upper portion with the lower portion; and
a control module received within the housing and adapted to index a count each time the dose indicator is cycled.

2. The dose indicator of claim 1, wherein the spring element includes a number of contacts, wherein the control module includes a number of contact pad pairs, wherein at least one contact pad pair is bridged by a corresponding contact when the dose indicator is in, a closed state, and wherein none of the contact pad pairs are bridged by their corresponding contact when the dose indicator is in a open state.

3. The dose indicator of claim 1, wherein a wall of the housing is structured to deflect a portion of the spring element.

4. The dose indicator of claim 3, wherein the wall of the housing includes a retaining slot having a spring retaining member structured to engage a portion of the spring element, and wherein the retaining slot includes a ramp structured to deflect the portion of the spring element.

5. The dose indicator of claim 1, wherein the control module includes a display structured to indicate the count.

6. The dose indicator of claim 1, further comprising:
a base portion connected to the lower portion; and
a breakaway member having a first end structured to couple with the base portion and having a second end structured to couple with a metered dose inhaler.

7. The dose indicator of claim 6, wherein the first end of the breakaway member is structured to releasably couple with the base portion and the second end of the breakaway member is structured to non-releasably couple with the metered dose inhaler.

8. The dose indicator of claim 7, further comprising an adhesive member having a first surface attached to the second end of the breakaway member and a second surface attachable to the metered dose inhaler.

9. The dose indicator of claim 6 wherein the count is representative of the number of doses of a medicament contained within the metered dose inhaler.

10. A drug delivery system, comprising:
a metered dose inhaler, comprising:
a body;
a canister insertable into the body, wherein the canister is adapted to hold a medicament therein, and wherein the canister includes a stem structured to cause the canister to release a dose of the medicament when the metered dose inhaler is fired; and
a dose indicator connectable to the canister without restricting airflow into the body and structured to indicate a count representative of an amount of medicament within the canister, the dose indicator comprising:
a housing having an upper portion and a lower portion;
a spring element structured to moveably connect the upper portion with the lower portion; and
a control module received within the housing and adapted to index the count each time the dose indicator is cycled.

11. The drug delivery system of claim 10, wherein the dose indicator is structured to cycle each time the metered dose inhaler is fired.

12. The drug delivery system of claim 10, wherein the spring element includes a number of contacts, wherein the control module includes a number of contact pad pairs, wherein at least one contact pad pair is bridged by a corresponding contact when the dose indicator is in a closed state, and wherein none of the contact pad pairs are bridged by their corresponding contact when the dose indicator is in a open state.

13. The drug delivery system of claim 10, wherein the controller is adapted to detect the cycling of the dose indicator, index the count in response to detecting that the dose indicator was cycled, and display the count.

14. The drug delivery system of claim 10, wherein a wall of the housing is structured to deflect a portion of the spring element when the dose indicator is cycled.

15. The drug delivery system of claim 10, wherein a wall of the housing includes a retaining slot having a spring retaining member structured to engage a portion of the spring element, and wherein the retaining slot includes a ramp structured to deflect the portion of the spring element.

16. The drug delivery system of claim 10, wherein the dose indicator further comprises:
a base portion connected to the lower portion; and
a breakaway member having a first end structured to couple with the base portion and having a second end structured to couple with the canister.

17. The drug delivery system of claim 16, wherein the first end of the breakaway member is structured to releasably couple with the base portion and the second end of the breakaway member is structured to non-releasably couple with the canister.

18. The drug delivery system of claim 17, further comprising an adhesive member having a first surface attached to the second end of the breakaway member and a second surface attachable to the metered dose inhaler.

19. A method for tracking an amount of medicament within in a canister for a metered dose inhaler, the method comprising:
programming a count representative of a number of doses of medicament within the canister into a dose indicator, wherein the dose indicator comprises:
a housing having an upper portion and a lower portion;
a spring element structured to moveably connect the upper portion with the lower portion; and
a control module received within the housing and adapted to index the count each time the dose indicator is cycled;
connecting the dose indicator to the canister;
detecting when the dose indicator is cycled; and
responsive to the detecting, indexing the count.

20. The method of claim 19, wherein further comprising structuring the dose indicator to cycle substantially each time that the metered dose inhaler is fired.

21. The method of claim 19, further comprising: generating a warning when the count is less than or equal to a predetermined threshold.

22. The method of claim 19, further comprising:
disconnecting the dose indicator from the canister when the count is less than or equal to the predetermined threshold;
removing the canister from the metered dose inhaler;
inserting a new canister into the metered dose inhaler; and
connecting the dose indicator to the new canister.
